(19) United States
(12) Patent Application Publication
(10) Pub. No.: US 2024/0216231 A1
(43) Pub. Date: Jul. 4, 2024

(54) HIGH-HARDNESS LIQUID CRYSTAL COSMETIC COMPOSITION HAVING ELASTIC TEXTURE AND METHOD FOR PREPARING SAME

(71) Applicant: COSMAX, INC., Gyeonggi-Do (KR)

(72) Inventors: Sun Young KIM, Suwon-si, Gyeonggi-do (KR); Hyen Joung PARK, Yongin-si, Gyeonggi-do (KR); Jin Gu JI, Suwon-si, Gyeonggi-do (KR); Myeong Sam PARK, Gwangjin-gu, Seoul (KR)

(73) Assignee: COSMAX, INC., Gyeonggi-Do (KR)

(21) Appl. No.: 18/288,508

(22) PCT Filed: Mar. 24, 2022
(86) PCT No.: PCT/KR2022/004154

§ 371 Date: Oct. 26, 2023

(30) Foreign Application Priority Data
Apr. 29, 2021 (KR) 10-2021-0055955

(51) Int. Cl. A61K 8/02; A61Q 19/00

(52) U.S. Cl. CPC A61K 8/0295; A61Q 19/007; A61K 2800/594

(57) ABSTRACT

The disclosure relates to a high-hardness liquid crystal cosmetic composition having an elastic texture, and a method of preparing the same. In a liquid crystal cosmetic composition according to an aspect, by selectively mixing with viscosity modifiers, a high-hardness liquid crystal cosmetic composition having an elastic texture may be formed, and by containing low-concentration oil, the liquid crystal cosmetic composition may have a refreshing and light feeling with a moisture feeling as compared with an existing liquid crystal cosmetic composition.

### TECHNICAL FIELD

The disclosure relates to a high-hardness liquid crystal cosmetic composition having an elastic texture, and a method of preparing the same.

### BACKGROUND ART

In general, a liquid crystal is in an intermediate state between a solid and a liquid and generally refers to a system having a lamellar structure formed in which, in organic molecules that have hydrophilic parts and lipophilic parts at the same time, the hydrophilic parts are arranged with each other in a uniform manner, and the lipophilic parts are arranged with each other in a uniform manner to form a plurality of layers. Since liquid crystals behave like liquids in terms of viscosity and fluidity, but exhibit unique properties such as solid properties in terms of scattering and reflecting light, the liquid crystals have a wide range of applications in various fields such as cosmetics, pharmaceuticals, food, and electronics. In the fields of cosmetics and pharmaceuticals, liquid crystals are generated at the interfaces during preparation of emulsions to stabilize the emulsions. By having a structure similar to that of the interstitial lipids of cell membranes, liquid crystals not only perform functions of forming a skin protective film and moisturizing, but also improve the storage stability of various water-soluble and oil-soluble medicinal components and are used as effective delivery media therefor.

Under this technical background, research is being actively conducted to diversify liquid crystal formulations (Korean Registered Patent No. 10-0713564). However, most of the liquid crystal formulations are still general O/W emulsified creams, and the diversity of formulations is insufficient.

### DISCLOSURE

### Technical Problem

An aspect provides a liquid crystal cosmetic composition including, with respect to 100 parts by weight of an entire composition: a liquid crystal former at a content of 5 parts by weight to 10 parts by weight; and a viscosity modifier at a content of 0.05 parts by weight to 1 part by weight.

Another aspect provides a method of preparing a liquid crystal cosmetic composition, the method including: adding a viscosity modifier to an aqueous phase part; and mixing a liquid crystal former with the aqueous phase part to which the viscosity modifier is added.

### Technical Solution

An aspect provides a liquid crystal cosmetic composition including, with respect to 100 parts by weight of an entire composition, a liquid crystal former at a content of 5 parts by weight to 10 parts by weight, and a viscosity modifier at a content of 0.05 parts by weight to 1 part by weight.

In an embodiment, the liquid crystal cosmetic composition may be a high-hardness liquid crystal cosmetic composition having a moisture feeling and elasticity.

The liquid crystal former may include at least one selected from the group consisting of higher aliphatic alcohol, a nonionic surfactant, and oil.

The higher aliphatic alcohol may be at least one selected from the group consisting of C12-C16 alcohol, C14-C22 alcohol, cetearyl alcohol, cetyl alcohol, stearyl alcohol, behenyl alcohol, and myristyl alcohol, but is not limited thereto.

The nonionic surfactant may be at least one selected from the group consisting of cetearyl glucoside, cetearyl olivate, sorbitan olivate, sorbitan palmitate, sucrose polystearate, C12-C20 alkyl glucoside, polyglyceryl-3 methylglucose distearate, and hydrogenated lecithin, but is not limited thereto.

The oil may be at least one selected from the group consisting of dicaprylyl carbonate, ethylhexyl palmitate, neopentyl glycol dicaprate, cetyl ethylhexanoate, dicaprylyl ether, pentaerythrityl tetraethylhexanoate, pentaerythrityl tetraisostearate, coco-caprylate/caprate, caprylic/capric triglyceride, and ethylhexyl stearate, but is not limited thereto.

The liquid crystal former may be contained at a content of 5 parts by weight to 10 parts by weight, preferably 7 parts by weight to 10 parts by weight, more preferably 7.5 parts by weight to 9.5 parts by weight, and even more preferably 8 parts by weight to 9 parts by weight, with respect to 100 parts by weight of the entire composition. In this case, when the content of the liquid crystal former is less than 5 parts by weight, a liquid crystal forming effect is not achieved, and when the content of the liquid crystal former exceeds 10 parts by weight, an oil phase is contained at a high proportion, and thus a liquid crystal structure disappears over time, which damages formulation stability.

The viscosity modifier may include at least two selected from the group consisting of an acrylate/C10-C30 alkyl acrylate crosspolymer, a hydroxyethyl acrylate/sodium acryloyldimethyl taurate copolymer, a carbomer, an ammonium acryloyldimethyl taurate/VP copolymer, a polyacrylate crosspolymer-11, and sodium polyacryloyldimethyl taurate. When only one type of viscosity modifier is used, a liquid crystal may not be formed, a feeling of use may be reduced, or formulation stability may be reduced.

The viscosity modifier may be a combination of the acrylate/C10-C30 alkyl acrylate crosspolymer and the carbomer. When only the acrylate/C10-C30 alkyl acrylate crosspolymer is used as the viscosity modifier, liquid crystal formation may not be clear, an elastic texture may be reduced, and less moisture feeling may be felt, which may reduce a feeling of use, or when the liquid crystal cosmetic composition is stored at high temperature for a month, hardness may be confirmed to decrease, which may damage formulation stability. When only the carbomer is used as the viscosity modifier, a stable emulsion and liquid crystal may not be formed due to high viscosity, the liquid crystal cosmetic composition may not be applied onto the skin due to too high hardness, or a separation phenomenon may be observed at room temperature and a high temperature, resulting in poor formulation stability. On the other hand, when the combination of the acrylate/C10-C30 alkyl acrylate crosspolymer and the carbomer is used as the viscosity modifier, a stable liquid crystal with a distinct shape may be formed, a highly-hardness cosmetic composition having an elastic texture may be formed, and formulation stability may be excellent at a high temperature and a low temperature for a long time.

A mixing weight ratio of the acrylate/C10-C30 alkyl acrylate crosspolymer to the carbomer may be in a range of (1 to 10):(1 to 10), (1 to 5):(1 to 5), (1 to 4):(1 to 4), (1 to 3):(1 to 3), or (1 to 2):(1 to 2).

The viscosity modifier may be contained at a content of 0.05 parts by weight to 1 part by weight, preferably 0.1 parts by weight to 1 part by weight, more preferably 0.3 parts by weight to 0.7 parts by weight, and even more preferably 0.4 parts by weight to 0.6 parts by weigh, with respect to 100 parts by weight of the entire composition. In this case, when the content of the viscosity modifier is less than 0.05 parts by weight, a thickening effect is not achieved, and when the content of the viscosity modifier exceeds 1 part by weight, it is difficult to ensure long-term stability of a formulation, a moisture feeling in the composition is reduced to reduce a feeling of use, and an elastic texture, which is a feature of the composition, disappears, which make it difficult to differentiate the composition from general balm-type formulations.

The composition may have a hardness of 80 dyne/cm² to 150 dyne/cm².

When the hardness is in such a range, an excellent feeling of use may be achieved when the liquid crystal cosmetic composition is applied onto the skin. When the hardness is less than 80 dyne/cm², flowability becomes excessive to damage the elasticity or resilience of a formulation, and thus it is difficult to feel an excellent feeling of use according to an elastic texture and a moisture feeling. When the hardness exceeds 150 dyne/cm², stirring is not easy, and thus it is difficult to prepare the liquid crystal cosmetic composition, and also the hardness is too high, and thus the liquid crystal cosmetic composition may not be applied onto the skin.

In addition, the cosmetic composition may additionally include components commonly added to cosmetic compositions, for example, typical auxiliaries and carriers such as antioxidants, stabilizers, solubilizers, vitamins, pigments, and fragrances.

The cosmetic composition may be prepared into any formulation commonly prepared in the art, for example, may be formulated into a solution, a suspension, an emulsion, a paste, a gel, a cream, a lotion, a powder, a soap, a surfactant-containing cleansing cream, oil, powder foundation, emulsion foundation, wax foundation, or a spray, but one or embodiments are not limited thereto. More specifically, the cosmetic composition may be prepared into a formulation of a lotion such as a softening lotion or a nourishing lotion, a milky lotion such as a facial lotion or a body lotion, a cream such as a nourishing cream, a moisturizing cream, or an eye cream, an essence, a cosmetic ointment, a spray, a gel, a pack, a sunscreen, makeup base, or liquid type, solid type, or spray type foundation. In this case, the cosmetic composition may include an emulsifier, a soapy acid, a solvent, a coloring agent, a preservative, an antioxidant, a defoamer, an antibacterial agent, an anti-redeposition agent, an enzyme, plant or mineral oil, fat, a fluorescent substance, a fungicide, a hydrotrope, a moisturizer, a flavoring agent, a flavoring agent carrier, a preservative, a protein, silicone, a solubilizer, a sugar derivative, a sunscreen, a vitamin, a plant extract, wax, and the like.

When the formulation of the cosmetic composition is a paste, a cream, or a gel, animal oil, vegetable oil, wax, paraffin, starch, tragacanth, a cellulose derivative, polyethylene glycol, silicone, bentonite, silica, talc, or zinc oxide may be used as a carrier component.

When the formulation of the cosmetic composition is a powder or a spray, toss, talc, silica, aluminum hydroxide, calcium silicate, or a polyamide powder may be used as a carrier component. In particular, when the formulation of the cosmetic composition is the spray, the carrier component may additionally include a propellant such as chlorofluorohydrocarbon, propane/butane, or dimethyl ether.

When the formulation of the cosmetic composition is a solution or an emulsion, a solvent, a solubilizer, or an emulsifier is used as a carrier component, and examples thereof include water, ethanol, isopropanol, ethyl carbonate, ethyl acetate, benzyl alcohol, benzyl benzoate, propylene glycol, 1,3-butyl glycol oil, glycerol aliphatic ester, or fatty acid ester of polyethylene glycol or sorbitan.

When the formulation of the cosmetic composition is a suspension, a liquid diluent such as water, ethanol, or propylene glycol, a suspension such as ethoxylated isostearyl alcohol, polyoxyethylene sorbitol ester, or polyoxyethylene sorbitan ester, microcrystalline cellulose, aluminum metahydroxide, bentonite, agar or tragacanth, and the like. the carrier component may be used as a carrier component.

According to a skin area onto which the cosmetic composition is to be applied, an appropriate amount of the cosmetic composition may be applied and used. The cosmetic composition may be repeatedly used from once to several times a day as needed. In addition, the amount and number of the application may be changed as needed according to skin condition and age of an individual.

Another aspect provides a method of preparing a liquid crystal cosmetic composition, the method including adding a viscosity modifier to an aqueous phase part, and
mixing a liquid crystal former with the aqueous phase part to which the viscosity modifier is added.

In an embodiment, the method of preparing a liquid crystal cosmetic composition is performed in the order of first adding the viscosity modifier to the aqueous phase part and then adding an oil phase to form a liquid crystal so that a prepared cosmetic composition is characterized by having a high hardness formulation. Through the above method, a high-hardness formulation may be implemented with only a viscosity modifier and a small amount of oil phase, and a prepared cosmetic composition has a lower wax and oil content as compared with general high-hardness formulations, and thus a formulation thereof is characterized by having an excellent elastic texture and also having a light feeling of use.

The liquid crystal former may include at least two selected from C12-C16 alcohol, C14-C22 alcohol, palmitic acid, hydrogenated lecithin, cetearyl alcohol, cetyl alcohol, stearyl alcohol, behenyl alcohol, myristyl alcohol, sorbitan olivate, sorbitan palmitate, caprylic/capric triglyceride, and ceramide NP, but one or more embodiments are not limited thereto.

The viscosity modifier may include at least two selected from an acrylate/C10-C30 alkyl acrylate crosspolymer, a hydroxyethyl acrylate/sodium acryloyldimethyl taurate copolymer, a carbomer, an ammonium acryloyldimethyl taurate/VP copolymer, a polyacrylate crosspolymer-11, and sodium polyacryloyldimethyl taurate, but one or more embodiments are not limited thereto.

The viscosity modifier may be a combination of the acrylate/C10-C30 alkyl acrylate crosspolymer and the carbomer.

The liquid crystal former may be contained at a content of 5 parts by weight to 10 parts by weight with respect to 100 parts by weight of the entire composition, and the viscosity modifier may be contained at a content of 0.05 parts by weight to 1 part by weight with respect to 100 parts by weight of the entire composition.

In addition, the method of preparing a liquid crystal cosmetic composition may further include adding a neutralizing agent to an emulsified emulsion formed by mixing the liquid crystal former with the aqueous phase part. The neutralizing agent may be, for example, tromethamine, but is not limited thereto, and any neutralizing agent commonly used in the art may be used without limitation.

Descriptions of parts of the viscosity modifier and the liquid crystal former, which overlap that of the liquid crystal cosmetic composition, will be omitted.

### Advantageous Effects

In a liquid crystal cosmetic composition according to an aspect, by selectively mixing and using viscosity modifiers, a high-hardness liquid crystal cosmetic composition having an elastic texture may be formed, and by containing low-concentration oil, the liquid crystal cosmetic composition may have a moisture feeling to have a refreshing and light feeling of use as compared with an existing liquid crystal cosmetic composition.

### DESCRIPTION OF DRAWINGS

FIG. 1 shows a result of showing whether a liquid crystal cosmetic composition according to an aspect forms a liquid crystal.

### BEST MODE

Hereinafter, the disclosure will be described in more detail through examples. However, these examples are for describing the disclosure in more detail, and the scope of the disclosure is not limited to these examples.

### EXAMPLES

### Examples 1 to 6: Preparation of High-Hardness Liquid Crystal Cosmetic Composition Having Elastic Texture

Examples were prepared to confirm an optimal mixing weight ratio of viscosity modifiers for a high-hardness cosmetic composition that included low-concentration oil to have a better moisture feeling and elastic appearance as compared with an existing liquid crystal cosmetic composition.

Cosmetic compositions of Examples 1 to 6 having compositions and contents shown in Table 1 below (unit: wt %) were prepared. A beaker containing raw materials to be included in an aqueous phase part was heated to a temperature of 70° C. to 80° C. to dissolve an aqueous phase component, and mixing was performed by using a homomixer (product name: Mark II manufactured by PRIMIX Corporation in Japan) until all components were uniformly dispersed. After the mixing of the aqueous phase part was completed, viscosity modifiers were added and uniformly dispersed. Afterwards, a beaker containing raw materials to be included in an oil phase part was heated to dissolve and mix the raw materials in the same manner as the aqueous phase part, and then mixing and emulsifying were performed by using a homomixer. Thereafter, a neutralizing agent was added to an emulsified emulsion to prepare a high-hardness liquid crystal cosmetic composition.

TABLE 1

| | | Example | Example | Example | Example | Example | Example |
|---|---|---|---|---|---|---|---|
| | | 1 | 2 | 3 | 4 | 5 | 6 |
| | Component name | Content (%) | | | | | |
| Aqueous | Purified water | 78.60 | 78.60 | 78.60 | 78.60 | 78.60 | 78.60 |
| phase part | Glycerin | 5.00 | 5.00 | 5.00 | 5.00 | 5.00 | 5.00 |
| | Butylene glycol | 5.00 | 5.00 | 5.00 | 5.00 | 5.00 | 5.00 |
| | Pentylene glycol | 2.00 | 2.00 | 2.00 | 2.00 | 2.00 | 2.00 |
| Viscosity | Acrylate/C10-C30 alkyl | 0.40 | 0.50 | 0.30 | 0.25 | 0.20 | — |
| modifier | acrylate crosspolymer | | | | | | |
| | Carbomer | 0.10 | — | 0.20 | 0.25 | 0.30 | 0.50 |
| Oil phase | Caprylic/capric | | | | | | |
| part | triglyceride | 5.00 | 5.00 | 5.00 | 5.00 | 5.00 | 5.00 |
| | Behenyl alcohol | 2.00 | 2.00 | 2.00 | 2.00 | 2.00 | 2.00 |
| | Cetearyl alcohol | 1.00 | 1.00 | 1.00 | 1.00 | 1.00 | 1.00 |
| | C14-C22 alcohol | 0.50 | 0.50 | 0.50 | 0.50 | 0.50 | 0.50 |
| | Ceramide NP | 0.10 | 0.10 | 0.10 | 0.10 | 0.10 | 0.10 |
| Neutralizing | Tromethamine | 0.30 | 0.30 | 0.30 | 0.30 | 0.30 | 0.30 |
| agent | | | | | | | |

### EXPERIMENTAL EXAMPLES

### Experimental Example 1: Confirmation of Formation of Liquid Crystal in Cosmetic Composition

It was confirmed whether the cosmetic compositions prepared in Examples 1 to 6 formed liquid crystals.

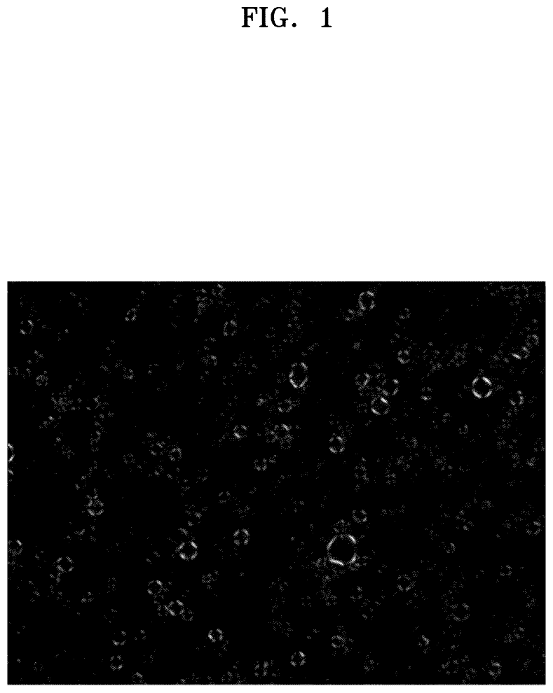

As a result, it was confirmed that the cosmetic composition of Example 1 formed stable liquid crystals having a distinct shape as shown in FIG. 1. In addition, the cosmetic compositions of Examples 3 to 5 also formed stable liquid crystals. In the case of Example 2, liquid crystal formation was not clearly visible. In the case of Example 6, viscosity formed due to the addition of the viscosity modifiers during the preparation was high, and thus stirring was not easy and a stable emulsion and liquid crystal were not formed.

### Experimental Example 2: Evaluation of Hardness and Feeling of Use of Cosmetic Composition

The hardness of the cosmetic compositions prepared in Examples 1 to 6 was measured after the cosmetic compositions were stored at room temperature for one day. The hardness was measured up to a depth of 25 mm of a sample at an operating speed of 2 cm/min by using Fudoh Rheometer RT-3005D (manufactured by Rheotech Co., Ltd.). After the hardness was measured 5 times, an average value was derived.

TABLE 2

| | Example 1 | Example 2 | Example 3 | Example 4 | Example 5 | Example 6 |
|---|---|---|---|---|---|---|
| Hardness (dyne/cm²) | 100 | 80 | 120 | 120 | 130 | — |
| Elastic force | ◯ | Δ | ◯ | ◯ | ◯ | — |

As a result, as shown in Table 2, it could be confirmed that the cosmetic compositions of Examples 1 and 3 to 5 were prepared as high-hardness cosmetic compositions having an elastic texture. In addition, although the cosmetic compositions had high hardness, the cosmetic compositions had a moderate elastic texture so that, when applied onto the skin, the cosmetic compositions had crush-like unique spreadability, a refreshing and light texture that felt moisturizing, was quickly absorbed, and provided an excellent overall feeling of use. On the other hand, in the case of Example 2 using only an acrylate/C10-C30 alkyl acrylate crosspolymer with emulsifying capacity among viscosity modifiers, it could be confirmed that the cosmetic composition has a lower elastic texture than the cosmetic compositions of Examples 1 and 3 to 5 and was somewhat thick and stiff during rolling like general creams, and thus it felts less hydrated. In addition, in the case of Example 6 using only a carbomer which had no emulsifying capacity and had high thickening power among viscosity modifiers, a large increase in viscosity of an emulsion when the emulsion was added, stirring was not easy, and thus a stable emulsion was not formed, and due to too high hardness, the cosmetic composition of Example 6 could not be applied onto the skin.

### Experimental Example 3: Evaluation of Stability of Cosmetic Composition

The stability of the cosmetic compositions prepared in Examples 1 to 6 was evaluated. While the cosmetic compositions were stored for 5 days, 10 days, 15 days, and one month under conditions of room temperature and a high temperature of 45° C., formulation stability was observed according to whether hardness decreased, and oil floating or separation occurred. Results thereof are shown in Table 3 below.

TABLE 3

| | Room temperature | | | | 45° C. | | | |
|---|---|---|---|---|---|---|---|---|
| | 5 days | 10 days | 15 days | One month | 5 days | 10 days | 15 days | One month |
| Example 1 | ◯ | ◯ | ◯ | ◯ | ◯ | ◯ | ◯ | ◯ |
| Example 2 | ◯ | ◯ | ◯ | ◯ | ◯ | ◯ | ◯ | Δ |
| Example 3 | ◯ | ◯ | ◯ | ◯ | ◯ | ◯ | ◯ | ◯ |
| Example 4 | ◯ | ◯ | ◯ | ◯ | ◯ | ◯ | ◯ | ◯ |
| Example 5 | ◯ | ◯ | ◯ | ◯ | ◯ | ◯ | ◯ | ◯ |

| | | |
|---|---|---|
| Example 6 | Occurrence of separation phenomenon | Occurrence of separation phenomenon |

As can be seen in Table 3, in the case of Example 2, a decrease in hardness was confirmed when the composition was stored at a high temperature for one month, and in Example 6, separation phenomena such as oil floating were confirmed at room temperature and high temperature, and formulation stability was not good. On the other hand, in Example 1 and Examples 3 to 5, it was confirmed that formulation stability was stably maintained irrespective of a storage temperature and period.

Through the above experimental results, it could be confirmed that, in a liquid crystal cosmetic composition according to an aspect, viscosity modifiers were selectively mixed and used, thereby forming a high-hardness liquid crystal cosmetic composition having an elastic texture and also securing an excellent feeling of use and high stability.

1. A liquid crystal cosmetic composition comprising, with respect to 100 parts by weight of an entire composition:
a liquid crystal former at a content of 5 parts by weight to 10 parts by weight; and
a viscosity modifier at a content of 0.05 parts by weight to 1 part by weight.

2. The liquid crystal cosmetic composition of claim 1, wherein the liquid crystal former comprises at least two selected from the group consisting of C12-C16 alcohol, C14-C22 alcohol, cetearyl alcohol, cetyl alcohol, stearyl alcohol, behenyl alcohol, myristyl alcohol, cetearyl glucoside, cetearyl olivate, sorbitan olivate, sorbitan palmitate, sucrose polystearate, C12-C20 alkyl glucoside, polyglyceryl-3 methylglucose distearate, hydrogenated lecithin, dicaprylyl carbonate, ethylhexyl palmitate, neopentyl glycol dicaprate, cetyl ethylhexanoate, dicaprylyl ether, pentaerythrityl tetraethylhexanoate, pentaerythrityl tetraisostearate, coco-caprylate/caprate, caprylic/capric triglyceride, and ethylhexyl stearate.

3. The liquid crystal cosmetic composition of claim 1, wherein the viscosity modifier comprises at least two selected from the group consisting of an acrylate/C10-C30 alkyl acrylate crosspolymer, a hydroxyethyl acrylate/sodium acryloyldimethyl taurate copolymer, a carbomer, an ammonium acryloyldimethyl taurate/VP copolymer, a polyacrylate crosspolymer-11, and sodium polyacryloyldimethyl taurate.

4. The liquid crystal cosmetic composition of claim 3, wherein the viscosity modifier comprises a combination of the acrylate/C10-C30 alkyl acrylate crosspolymer and the carbomer.

5. The liquid crystal cosmetic composition of claim 1, wherein the liquid crystal cosmetic composition has a hardness of 80 dyne/cm² to 150 dyne/cm².

6. The liquid crystal cosmetic composition of claim 1, wherein the liquid crystal cosmetic composition is in the form of a moisturizing cream having an elastic texture.

7. A method of preparing a liquid crystal cosmetic composition, the method comprising:
adding a viscosity modifier to an aqueous phase part; and
mixing a liquid crystal former with the aqueous phase part to which the viscosity modifier is added.

8. The method of claim 7, wherein the liquid crystal former comprises at least two selected from the group consisting of C12-C16 alcohol, C14-C22 alcohol, cetearyl alcohol, cetyl alcohol, stearyl alcohol, behenyl alcohol, myristyl alcohol, cetearyl glucoside, cetearyl olivate, sorbitan olivate, sorbitan palmitate, sucrose polystearate, C12-C20 alkyl glucoside, polyglyceryl-3 methylglucose distearate, hydrogenated lecithin, dicaprylyl carbonate, ethylhexyl palmitate, neopentyl glycol dicaprate, cetyl ethylhexanoate, dicaprylyl ether, pentaerythrityl tetraethylhexanoate, pentaerythrityl tetraisostearate, coco-caprylate/caprate, caprylic/capric triglyceride, and ethylhexyl stearate.

9. The method of claim 7, wherein the viscosity modifier comprises at least two selected from the group consisting of an acrylate/C10-C30 alkyl acrylate crosspolymer, a hydroxyethyl acrylate/sodium acryloyldimethyl taurate copolymer, a carbomer, an ammonium acryloyldimethyl taurate/VP copolymer, a polyacrylate crosspolymer-11, and sodium polyacryloyldimethyl taurate.

10. The method of claim 7, wherein the liquid crystal former is contained at a content of 5 parts by weight to 10 parts by weight with respect to 100 parts by weight of the entire composition, and the viscosity modifier is contained at a content of 0.05 parts by weight to 1 part by weight with respect to 100 parts by weight of the entire composition.
